(19) United States
(12) Patent Application Publication
(10) Pub. No.: US 2021/0378764 A1
(43) Pub. Date: Dec. 9, 2021

(54) CENTRALIZED CONTROL APPARATUS, SETTING METHOD, AND CENTRALIZED CONTROL SYSTEM

(71) Applicant: OLYMPUS CORPORATION, Tokyo (JP)

(72) Inventor: Yorito MAEDA, Tokyo (JP)

(73) Assignee: OLYMPUS CORPORATION, Tokyo (JP)

(21) Appl. No.: 17/408,793

(22) Filed: Aug. 23, 2021

Related U.S. Application Data
(63) Continuation of application No. PCT/JP2019/007080, filed on Feb. 25, 2019.

(51) Int. Cl. A61B 34/00; G06F 11/34

(52) U.S. Cl. CPC A61B 34/25; G06F 11/3476; A61B 2034/258; A61B 2034/254; G06F 11/3419

(57) ABSTRACT

A centralized controller includes a recording unit, a judgement unit, a processing unit and an execution unit. The judgement unit reads out information on a plurality of kinds of operation from the recording unit and performs scene correspondence judgement as to whether or not each of the plurality of kinds of operation is operation corresponding to the scene for each of scenes of a surgery defined in advance. The processing unit generates at least part of setting values of a plurality of pieces of controlled equipment from a plurality of kinds of operation which are respectively judged as the operation corresponding to the scene through the scene correspondence judgement and records the setting values in the recording unit. The execution unit collectively sets the setting values for one or more pieces of controlled equipment for each of the scenes of the surgery.

### CROSS REFERENCE TO RELATED APPLICATION

This application is a continuation application of PCT/JP2019/007080 filed on Feb. 25, 2019, the entire contents of which are incorporated herein by this reference.

### BACKGROUND OF THE INVENTION

### 1. Field of the Invention

The present invention relates to a centralized control apparatus, a setting method, and a centralized control system, and, more particularly, to a centralized control apparatus which is capable of collectively setting values at a plurality of pieces of controlled equipment, a setting method and a centralized control system.

### 2. Description of the Related Art

Various kinds of equipment for medical use and for non-medical use are provided within a surgery room. Various kinds of equipment include a shadowless light, an endoscope apparatus, an insufflation apparatus, an electrocautery apparatus, and the like. During a surgery, setting of ON/OFF, setting, change, or the like, of output setting values are performed on various kinds of equipment for each scene of the surgery. Such operation on various kinds of equipment can be performed in a centralized manner by using a centralized controller as a centralized control apparatus.

The centralized controller has a function of collectively setting values of various kinds of equipment for each scene of a surgery. The setting values of various kinds of equipment are registered in advance at the centralized controller. The function enables prompt setting of various kinds of equipment. Further, the centralized controller has a function of recording operation history of various kinds of equipment as operation log information. A user, for example, registers the setting values of various kinds of equipment with reference to the operation log information.

Japanese Patent Application Laid-Open Publication No. 2007-68564 discloses a surgery system which sets medical equipment and sets non-medical equipment based on setting information set in advance. Japanese Patent Application Laid-Open Publication No. 2006-223375 discloses a system controller which records surgery data in association with predetermined time and records event data in association with the predetermined time in accordance with occurrence of a predetermined event.

International Publication No. 2015/087612 discloses a control apparatus which controls a plurality of pieces of medical equipment and which collectively sets the plurality of pieces of medical equipment using operation setting values read out from a storage unit. The operation setting values are stored in the storage unit in association with a plurality of scene items.

### SUMMARY OF THE INVENTION

A centralized control apparatus according to one aspect of the present invention is a centralized control apparatus including a processor, the processor being configured to record information on a plurality of kinds of operation performed on a plurality of pieces of controlled equipment along with time points at which the plurality of kinds of operation are performed and record the information on the plurality of kinds of operation as one piece of operation log information for each piece of specific information which specifies at least one of a name of a surgeon or a name of procedure, read out the piece of the operation log information in plurality from a recording result of the operation log information for each piece of the specific information, extract an aggregate of a plurality of kinds of operation which are included in different pieces of the operation log information and to which the same operation belongs as one scene candidate aggregate and extract the scene candidate aggregate in plurality, in a case where each of the plurality of kinds of operation belonging to the scene candidate aggregate is set as each of trigger operation candidates from an extraction result of the scene candidate aggregate, count, for each of the scene candidate aggregate in plurality, the number of the trigger operation candidates which correspond to the same operation belonging to the scene candidate aggregate, determine, for each of the scene candidate aggregate in plurality, one piece of trigger operation associated with the scene candidate aggregate from a plurality of the trigger operation candidates belonging to the scene candidate aggregate based on the counted number of the trigger operation candidates and record the trigger operation as the operation log information in association with the specific information, and read out setting values of one or more pieces of controlled equipment which are setting targets based on the operation log information for each of scenes of a surgery defined in advance and collectively set the setting values at the one or more pieces of controlled equipment.

A setting method according to one aspect of the present invention is a setting method in which a centralized control apparatus sets setting values at a plurality of pieces of controlled equipment in accordance with scenes of a surgery, the setting method including recording information on a plurality of kinds of operation along with time points at which the plurality of kinds of operation are performed and recording the information on the plurality of kinds of operation as one piece of operation log information including a plurality of aggregates each including the plurality of kinds of operation which are continuous on a time-series basis and for which an interval between two kinds of operation which are adjacent to each other on the time-series basis is equal to or less than a predetermined time period, for each of specific information which specifies at least one of a name of a surgeon or a name of procedure, reading out the piece of operation log information in plurality for each piece of the specific information, extracting an aggregate of a plurality of kinds of operation which are included in different pieces of the operation log information and to which the same operation belongs as one scene candidate aggregate and extracting the scene candidate aggregate in plurality, in a case where each of the plurality of kinds of operation belonging to the scene candidate aggregate is set as each of trigger operation candidates, counting, for each of the scene candidate aggregate in plurality, the number of the trigger operation candidates which correspond to the same operation belonging to the scene candidate aggregate, determining, for each of the scene candidate aggregate in plurality, one piece of trigger operation associated with the scene candidate aggregate from a plurality of the trigger operation candidates belonging to the scene candidate aggregate based on the counted number of the trigger operation candidates and recording the trigger operation as the operation log information in association with the specific information, and reading out setting values of one or more pieces of controlled equipment which are setting targets based on the operation log information for each of scenes of the surgery defined in advance and collectively setting the setting values at the one or more pieces of controlled equipment.

A centralized control system according to one aspect of the present invention includes a plurality of pieces of controlled equipment, and a processor to which the plurality of pieces of controlled equipment are connected, the processor being configured to record information on a plurality of kinds of operation along with time points at which the plurality of kinds of operation are performed and record the information on the plurality of kinds of operation as one piece of operation log information for each piece of specific information which specifies at least one of a name of a surgeon or a name of procedure, read out the piece of the operation log information in plurality from a recording result of the operation log information for each piece of the specific information, extract an aggregate of a plurality of kinds of operation which are included in different pieces of the operation log information and to which the same operation belongs as one scene candidate aggregate and extract the scene candidate aggregate in plurality, in a case where each of the plurality of kinds of operation belonging to the scene candidate aggregate is set as each of trigger operation candidates from an extraction result of the scene candidate aggregate, count, for each of the scene candidate aggregate in plurality, the number of the trigger operation candidates which correspond to the same operation belonging to the scene candidate aggregate, determine, for each of the scene candidate aggregate in plurality, one piece of trigger operation associated with the scene candidate aggregate from a plurality of the trigger operation candidates belonging to the scene candidate aggregate based on the counted number of the trigger operation candidates and record the trigger operation as the operation log information in association with the specific information, and read out setting values of one or more pieces of controlled equipment which are setting targets based on the operation log information for each of scenes of a surgery defined in advance and collectively set the setting values at the one or more pieces of controlled equipment.

### BRIEF DESCRIPTION OF THE DRAWINGS

FIG. 1 is an explanatory diagram illustrating a configuration of a surgery system in a first embodiment of the present invention;

FIG. 2 is a functional block diagram illustrating a configuration of a centralized controller according to the first embodiment of the present invention;

FIG. 3 is an explanatory diagram illustrating an example of a hardware configuration of the centralized controller according to the first embodiment of the present invention;

FIG. 4 is a flowchart illustrating setting value generation processing in the first embodiment of the present invention;

FIG. 5 is an explanatory diagram illustrating an example of operation log information in the first embodiment of the present invention:

FIG. 6 is an explanatory diagram illustrating an example of a setting screen for setting a registration condition for registering collective settings in the first embodiment of the present invention;

FIG. 7 is an explanatory diagram illustrating another example of the setting screen for setting the registration condition for registering collective settings in the first embodiment of the present invention;

FIG. 8 is an explanatory diagram illustrating a setting screen for setting a scene condition for registering collective settings in the first embodiment of the present invention;

FIG. 9 is an explanatory diagram illustrating a setting screen for setting trigger operation in the first embodiment of the present invention;

FIG. 10 is a functional block diagram illustrating a configuration of a centralized controller according to a second embodiment of the present invention;

FIG. 11 is a flowchart illustrating trigger operation determination processing in the second embodiment of the present invention; and

FIG. 12 is a flowchart illustrating setting value generation processing in the second embodiment of the present invention.

### DETAILED DESCRIPTION OF THE PREFERRED EMBODIMENTS

Embodiments of the present invention will be described below with reference to the drawings.

### First Embodiment

### (Configuration of Surgery System)

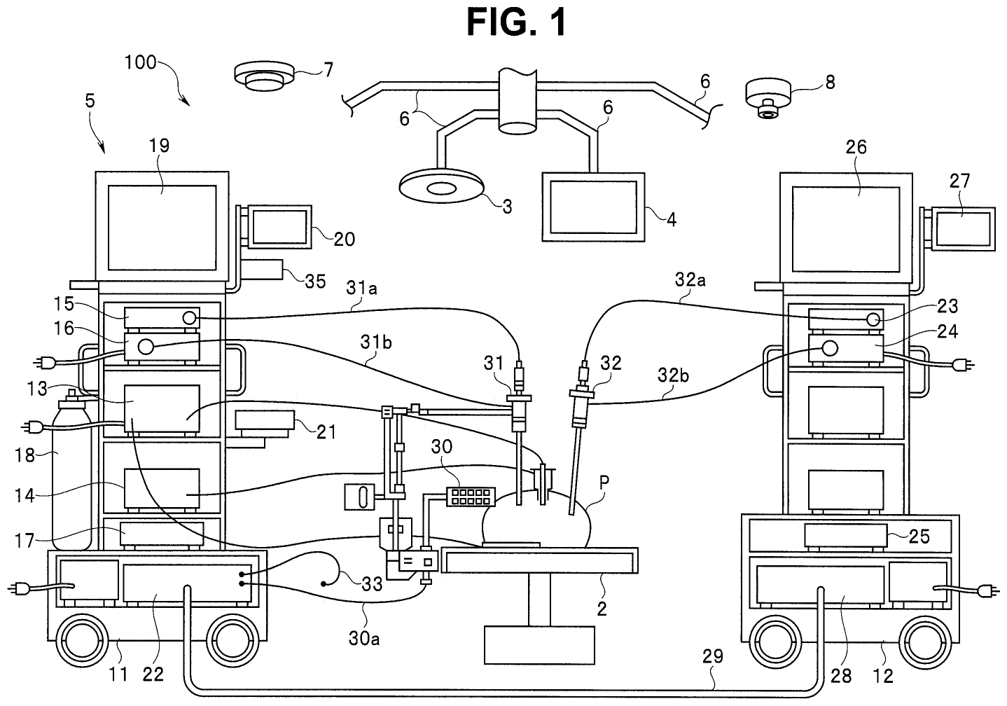

An entire configuration of a surgery system 100 including a centralized control apparatus according to a first embodiment of the present invention will be described first. FIG. 1 is an explanatory diagram illustrating a configuration of the surgery system 100. The surgery system 100 includes a plurality of pieces of medical equipment such as an electrocautery apparatus and a plurality of pieces of non-medical equipment such as a room light.

As illustrated in FIG. 1, a surgical table 2 on which a patient P lies down, a plurality of shadowless lights 3, a display apparatus 4, and a medical system 5 are disposed within a surgery room. The plurality of shadowless lights 3 and the display apparatus 4 are fixed on a ceiling of the surgery room with arms 6. Further, a room light 7, a surgical field camera 8, and a room camera which is not illustrated are provided within the surgery room.

The medical system 5 includes a first cart 11 and a second cart 12. On the first cart 11, a plurality of pieces of equipment such as an electrocautery apparatus 13, an insufflation apparatus 14, a video system center 15, a light source apparatus 16 and a recorder 17 for video recording, and a gas cylinder 18 filled with carbon dioxide are placed as medical equipment. Note that while not illustrated in FIG. 1, an ultrasound coagulation dissection apparatus may be further placed on the first cart 11 as medical equipment.

The video system center 15 is connected to a first endoscope 31 via a camera cable 31a. The light source apparatus 16 is connected to the first endoscope 31 via a light guide cable 31b. The recorder 17 is a recording apparatus including a large-capacity storage apparatus such as a hard disk apparatus.

Further, a display apparatus 19, a first centralized display panel 20, and an operation panel apparatus 21 are placed on the first cart 11. The display apparatus 19, which is an apparatus displaying an endoscope image, or the like, picked up by the first endoscope 31, is, for example, configured with a TV monitor. The centralized display panel 20 is display means which can cause every kind of data to be selectively displayed during a surgery.

The operation panel apparatus 21 is a centralized operation apparatus to be used by a nurse, or the like, in an unsterilized area to operate each piece of equipment, or the like. The operation panel apparatus 21 includes, for example, a display unit such as a liquid crystal display, and a touch panel integrally provided on the display unit.

A centralized controller 22 which is a centralized control apparatus according to the present embodiment is further placed on the first cart 11. A plurality of pieces of equipment including the plurality of shadowless lights 3, the room light 7, the electrocautery apparatus 13, the insufflation apparatus 14, the video system center 15, the light source apparatus 16 and the recorder 17 are connected to the centralized controller 22 via a communication line which is not illustrated.

Further, a headset-type microphone 33 is connected to the centralized controller 22. The centralized controller 22 is configured so as to be able to recognize voice inputted from the microphone 33 and control respective pieces of equipment with the voice of a surgeon.

Further, an RFID (radio frequency identification) terminal 35 is provided on the first cart 11. The RFID terminal 35 is an apparatus which reads individual ID information of an object from an ID tag embedded into treatment instrument, or the like, of the first endoscope 31 and the electrocautery apparatus 13 and writes necessary information in the ID tag.

A plurality of pieces of equipment such as a video system center 23, a light source apparatus 24, an image processing apparatus 25, a display apparatus 26 and a centralized display panel 27 are placed on the second cart 12. The video system center 23 is connected to a second endoscope 32 via a camera cable 32a. The light source apparatus 24 is connected to the second endoscope 32 via a light guide cable 32b.

The display apparatus 26, which is an apparatus displaying an endoscope image, or the like, picked up by the second endoscope 32, is, for example, configured with a TV monitor. The centralized display panel 27 is display means which can cause every kind of data to be selectively displayed during a surgery.

Further, a relay unit 28 is placed on the first cart 11. A plurality of pieces of equipment including the video system center 23, the light source apparatus 24 and the image processing apparatus 25 are connected to the relay unit 28 via a communication line which is not illustrated.

Further, the relay unit 28 is connected to the centralized controller 22 placed on the first cart 11 via a relay cable 29. With the configuration, the centralized controller 22 can control a plurality of pieces of equipment including the plurality of shadowless lights 3, the room light 7, the electrocautery apparatus 13, the insufflation apparatus 14, the video system center 15, the light source apparatus 16 and the recorder 17 connected via a communication line which is not illustrated and a plurality of pieces of equipment including the video system center 23, the light source apparatus 24 and the image processing apparatus 25 connected to the relay unit 28 via a communication line which is not illustrated, in a centralized manner. Hereinafter, equipment which is controlled by the centralized controller 22 in a centralized manner among a plurality of pieces of equipment placed within the surgery room will also be referred to as controlled equipment.

Further, the centralized controller 22 is configured to be able to display a setting screen including setting states, operation switches, or the like, of a plurality of pieces of equipment which are connected to the centralized controller 22 and the relay unit 28, and for which communication with the centralized controller 22 is established on a display unit of the operation panel apparatus 21. Operation input such as change of setting values of the plurality of pieces of equipment can be performed by a predetermined area within the setting screen being touched via the touch panel of the operation panel apparatus 21.

Further, an infrared communication port which is not illustrated and which is communication means is connected to the centralized controller 22 via a communication cable which is not illustrated. The infrared communication port is provided at a position which is likely to be irradiated with infrared light, in the vicinity, or the like, of the display apparatus 19.

A remote controller 30 is further disposed within the surgery room. The remote controller 30 is connected to the centralized controller 22 via a communication cable 30a. The remote controller 30, which is a second centralized operation apparatus to be operated by a surgeon in a sterilized area, is configured so as to be able to operate a plurality of pieces of equipment for which communication with the centralized controller 22 is established, via the centralized controller 22.

### (Configuration of Centralized Controller)

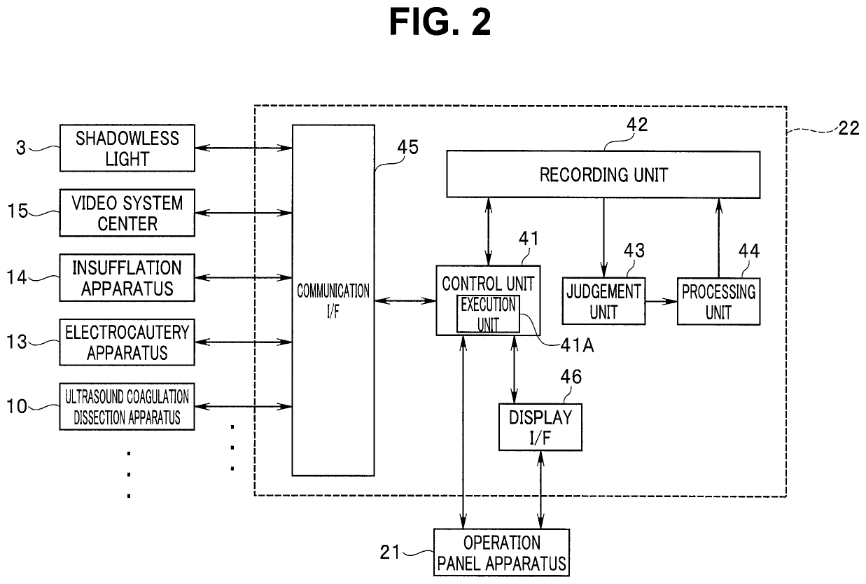

A configuration of the centralized controller 22 will be described in detail next with reference to FIG. 2. FIG. 2 is a functional block diagram illustrating the configuration of the centralized controller 22. The centralized controller 22 includes a control unit 41, a recording unit 42, a judgement unit 43, a processing unit 44, a communication interface (hereinafter, described as a communication I/F) 45, and a display interface (hereinafter described as a display I/F) 46.

The control unit 41 controls respective components within the centralized controller 22 to implement various kinds of functions of the centralized controller 22, more specifically, a function of controlling operation of a plurality of pieces of controlled equipment, a function of executing collective setting for each scene of the surgery, a function of generating various kinds of screens such as an operation screen for a designated function, a function of recording operation log information, or the like.

A plurality of communication lines connected to a plurality of pieces of controlled equipment are connected to the communication I/F 45. FIG. 2 illustrates the shadowless light 3, the ultrasound coagulation dissection apparatus 10, the electrocautery apparatus 13, the insufflation apparatus 14 and the video system center 15 among the plurality of pieces of controlled equipment. The communication I/F 45 is an interface circuit to be used by the control unit 41 to perform communication with a plurality of pieces of controlled equipment directly or indirectly connected to the centralized controller 22. The control unit 41 can turn ON/OFF the plurality of pieces of controlled equipment, set and change the setting values, and acquire operation states, or the like, by performing communication with the plurality of pieces of controlled equipment via the communication I/F 45.

Further, the control unit 41 receives an operation signal from the operation panel apparatus 21 which is an operation apparatus connected to the centralized controller 22 and outputs an image signal of a screen to be displayed at the display unit of the operation panel apparatus 21 to the operation panel apparatus 21 via the display I/F 46.

A login screen for causing the centralized controller 22 to operate and an operation screen for executing functions of the plurality of pieces of controlled equipment are displayed at the display unit of the operation panel apparatus 21. A surgeon, a nurse, or the like, (hereinafter, referred to as a user), for example, logs in the centralized controller 22 by performing touch operation on a selection screen of a surgeon and procedure displayed on the login screen. The surgeon corresponds to a login name. Further, the user can give an instruction to execute the functions of the plurality of pieces of controlled equipment or can set and change the setting values of the plurality of piece of controlled equipment by performing touch operation on various kinds of operation buttons displayed on the operation screen.

Further, a screen for selecting a scene to make collective settings on the plurality of pieces of controlled equipment is displayed at the display unit of the operation panel apparatus 21. The user can give an instruction to execute collective settings by selecting a scene.

Further, the user selects a scene and performs a surgery while directly operating the plurality of pieces of controlled equipment or indirectly operating the plurality of pieces of controlled equipment via the operation panel apparatus 21. The recording unit 42 records information on a plurality of kinds of operation directly performed on the plurality of pieces of controlled equipment or indirectly performed via the operation panel apparatus 21 as the operation log information. In the present embodiment, the recording unit 42 records the above-described information along with time points at which the plurality of kinds of operation are performed. Note that the operation on the controlled equipment is, more specifically, turning ON/OFF of the equipment, setting and change of output values, thresholds, or the like.

Further, the recording unit 42 records the operation log information in the centralized controller 22 in association with login information. The login information includes specific information which specifies at least one of a name of a surgeon or a name of procedure. In the present embodiment, the recording unit 42 records the operation log information in association with the name of the surgeon and the name of the procedure. In other words, the recording unit 42 records information on the plurality of kinds of operation as one piece of operation log information for each name of the surgeon and for each name of the procedure.

Further, a screen for setting the setting values of the plurality of pieces of controlled equipment for each scene of a surgery is displayed at the display unit of the operation panel apparatus 21. The setting values are, for example, ON/OFF of the equipment, output values, thresholds, or the like. The user can set the setting values by inputting desired values in input fields of the setting values displayed on the screen. Further, the user can give an instruction to execute processing of automatically generating setting values (hereinafter, referred to as setting value generation processing) by, for example, touching a button for executing setting value generation. The control unit 41 controls the judgement unit 43 and the processing unit 44 to generate setting values in a case where a signal which gives an instruction to execute the setting value generation processing is input from the operation panel apparatus 21.

The judgement unit 43 reads out the operation log information from the recording unit 42 and performs scene correspondence judgement as to whether or not each of a plurality of kinds of operation included in the operation log information is operation corresponding to the scene, which is operation of the scene, for each of scenes of a surgery defined in advance.

The processing unit 44 generates at least part of the setting values of the plurality of pieces of controlled equipment in association with the scenes of the surgery from the plurality of kinds of operation which are respectively judged as the operation corresponding to the scene through the scene correspondence judgement and records the setting values in the recording unit 42. The processing unit 44 may execute the above-described processing every time the judgement unit 43 performs the scene correspondence judgement or may execute the above-described judgement after the judgement unit 43 performs the scene correspondence judgment for all scenes of the surgery.

The control unit 41 includes an execution unit 41A which reads out setting values of one or more pieces of controlled equipment which are setting targets from the recording unit 42 for each of the scenes of the surgery and collectively sets the setting values at one or more pieces of controlled equipment. The execution unit 41A executes the above-described processing in a case where a signal which gives an instruction to execute collective setting is input to the control unit 41 from the operation panel apparatus 21. At least part of the setting values to be set by the execution unit 41A are the setting values generated by the processing unit 44. Note that setting values other than the setting values generated by the processing unit 44, which are to be set by the execution unit 41A, are setting values registered in advance by the user.

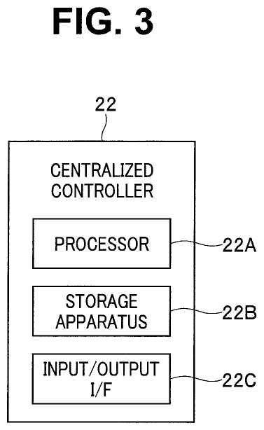

Here, a hardware configuration of the centralized controller 22 will be described with reference to FIG. 3. FIG. 3 is an explanatory diagram illustrating an example of the hardware configuration of the centralized controller 22. In the example illustrated in FIG. 3, the centralized controller 22 includes a processor 22A, a storage apparatus 22B and an input/output I/F 22C. The processor 22A is, for example, configured with a central processing unit (hereinafter, described as a CPU). The storage apparatus 22B is, for example, configured with a storage apparatus such as a RAM, a ROM, a flash memory and a hard disk apparatus. The input/output I/F 22C is used by the centralized controller 22 to transmit/receive a signal to/from outside and includes the above-described communication I/F 45 and the display I/F 46.

The processor 22A is used to execute functions of the control unit 41, the judgement unit 43, the processing unit 44, or the like, which are components of the centralized controller 22. The storage apparatus 22B stores a plurality of software programs for the functions. The respective functions are implemented by the processor 22A reading out a predetermined software program from the storage apparatus 22B and executing the software program. The storage apparatus 22B stores a control program for controlling operation of a plurality of pieces of controlled equipment, a setting value generation program for generating setting values of the plurality of pieces of controlled equipment for each scene of a surgery, a collective setting processing program for executing collective setting for each scene of a surgery, an image generation program for generating various kinds of screens such as an operation screen for a designated function, a history information recording program for recording the operation log information, and the like, as the plurality of software programs.

A function of the recording unit 42 which is a component of the centralized controller 22 is implemented by a non-volatile rewritable storage apparatus such as a flash memory and a hard disk apparatus among the storage apparatus 22B. The above-described non-volatile rewritable storage apparatus stores the operation log information and the setting values of the plurality of pieces of controlled equipment.

Particularly, the operation log information is stored in a predetermined saving folder created at the above-described non-volatile rewritable storage apparatus as a log file. The log file may be one file or a plurality of files which are divided for each name of the surgeon and each name of the procedure. In the latter case, each of the plurality of files may be stored in a saving folder corresponding to the name of the surgeon and the name of the procedure associated with the file.

Note that the hardware configuration of the centralized controller 22 is not limited to the above-described example. For example, the processor 22A may be configured with an FPGA (field programmable gate array). In the present case, at least part of the plurality of components of the centralized controller 22 are configured as circuit blocks in the FPGA. Alternatively, the plurality of components of the centralized controller 22 may be respectively configured as different electronic circuits.

### (Setting Value Generation Processing)

The setting value generation processing, that is, operation of the judgement unit 43 and the processing unit 44 will be described next. The operation of the judgement unit 43 will be conceptually described first. The judgement unit 43 reads out the operation log information (log file) recorded in the recording unit 42 and performs scene correspondence judgement as to whether or not each kind of a plurality of kinds of operation included in the operation log information is operation corresponding to the scene which is operation of the scene, for each scene of the surgery. The plurality of kinds of operation include a plurality of kinds of trigger operation associated with scenes of the surgery, which are different from each other. In the present embodiment, the trigger operation is registered in advance by the user. The judgement unit 43 performs scene correspondence judgement for each scene of the surgery based on a trigger time point which is a time point at which operation associated with the scene among the plurality of kinds of trigger operation is performed.

The scene correspondence judgement will be more specifically described below. First, a first judgement method will be described. In the first judgement method, the judgement unit 43 judges a plurality of kinds of operation (including trigger operation) performed within a predetermined time period including the trigger time point as the operation corresponding to the scene.

A second judgement method will be described next. In the second judgement method, the judgement unit 43 judges a plurality of kinds of operation (including trigger operation) which are continuous with the trigger operation on a time-series basis and for which an interval between two kinds of operation which are adjacent on a time-series basis is equal to or less than a predetermined time period, as the operation corresponding to the scene.

In the second judgement method, a plurality of aggregates which respectively correspond to different scenes of the surgery and which include one or more kinds of operation corresponding to the scene line on a time-series basis. Among the plurality of aggregates, two aggregates which are adjacent on a time-series basis will be referred to as a first aggregate and a second aggregate. An interval between a time point at which operation which is the closest to the second aggregate on a time-series basis in the first aggregate is performed and a time point at which operation which is the closest to the first aggregate on a time-series basis in the second aggregate is performed is greater than the predetermined time period. The predetermined time period is also a criterion for judging whether or not two kinds of operation which are adjacent on a time-series basis are operation of different scenes.

The operation of the judgement unit 43 and the processing unit 44 will be more specifically described next. Here, description will be provided using an example of laparoscopic cholecystectomy.

First, scenes of a surgery and trigger operation will be described. The scenes of the surgery and the trigger operation are registered in advance by the user. More specifically, a scene of preparation before the surgery, a scene of start of the surgery, a scene of insertion of an endoscope, a scene of closure and a scene of end of the surgery are registered as the scenes of the surgery.

Further, operation of logging in to the centralized controller 22 is registered as trigger operation associated with the scene of preparation before the surgery. The operation is registered as the trigger operation because typically preparation before the surgery is performed at a timing of login.

Further, operation of turning on the shadowless lights 3 is registered as trigger operation associated with the scene of start of the surgery. The operation is registered as the trigger operation because while it is necessary to dissect the skin to form a port first in the surgery, at the time, the shadowless lights 3 are necessarily turned on.

Further, operation of starting air feeding to the insufflation apparatus 14 is registered as trigger operation associated with the scene of insertion of an endoscope. The operation is registered as the trigger operation because while it is necessary to inflate a body cavity to insert the endoscope, at the time, air feeding to the insufflation apparatus 14 is necessarily started.

Further, operation of stopping air feeding to the insufflation apparatus 14 is registered as trigger operation associated with the scene of closure. The operation is registered as the trigger operation because in a case where closure is performed, it is not necessary to inflate the body cavity and air feeding to the insufflation apparatus 14 is necessarily stopped.

Further, operation of turning off the shadowless lights 3 is registered as trigger operation associated with the scene of end of the surgery. The operation is registered as the trigger operation because in a case where the surgery ends, the shadowless lights 3 are necessarily turned off.

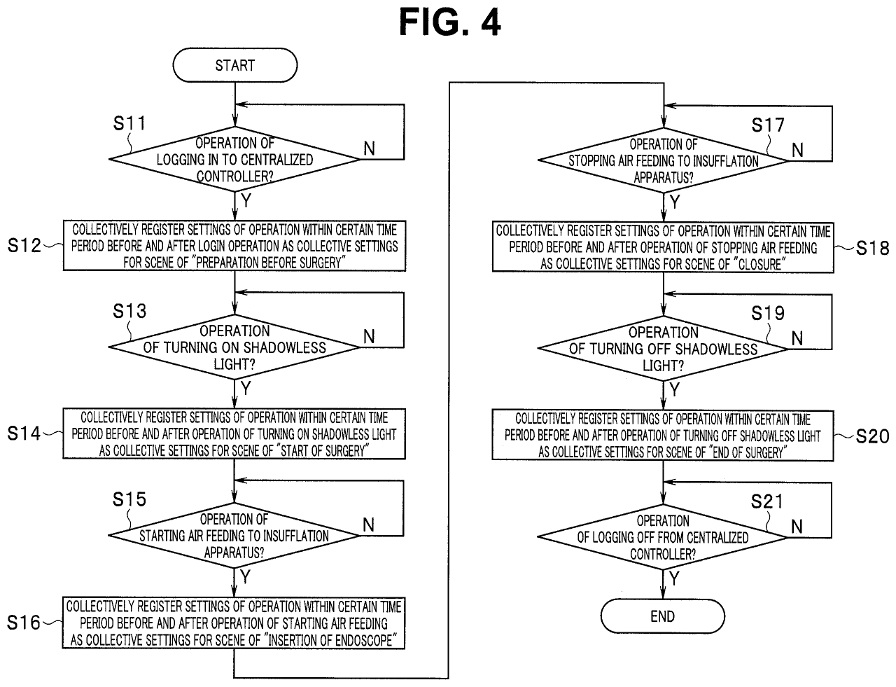

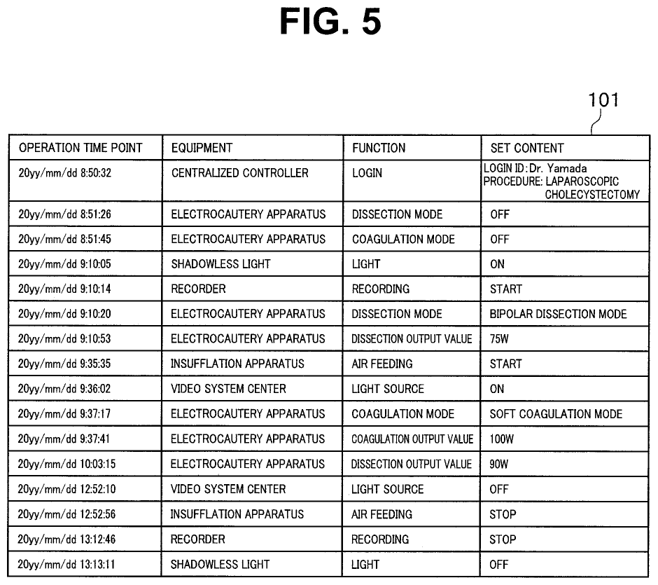

The setting value generation processing will be more specifically described below with reference to FIG. 4 and FIG. 5. FIG. 4 is a flowchart illustrating the setting value generation processing. FIG. 5 is an explanatory diagram illustrating an example of the operation log information. Here, description will be provided using an example of a case where the setting value generation processing is performed on the operation log information 101 illustrated in FIG. 5. Further, here, description will be provided using an example of a case where the judgement unit 43 performs scene correspondence judgement using the above-described first judgement method.

As illustrated in FIG. 4, in the setting value generation processing, first, the judgement unit 43 reads out the operation log information 101, reads out first operation on a time-series basis, and judges whether or not the readout operation is operation of logging in to the centralized controller 22 (step S11). In a case where the readout operation is not the login operation (N), the judgement unit 43 reads out operation which is next on a time-series basis and executes step S11 again. In a case where the readout operation is the login operation (Y), settings of operation within a certain time period before and after the login operation are collectively registered as collective settings for the scene of “preparation before the surgery” (step S12). More specifically, the judgement unit 43 judges operation of turning off a dissection mode of the electrocautery apparatus 13 (operation time point: 8:51:26) and operation of turning off a coagulation mode of the electrocautery apparatus 13 (operation time point: 8:51:45) as operation corresponding to the scene of “preparation before the surgery”. Then, the processing unit 44 generates settings of turning off the dissection mode of the electrocautery apparatus 13 and setting of turning off the coagulation mode of the electrocautery apparatus 13 in association with the scene of “preparation before the surgery” and records the settings in the recording unit 42.

In the setting value generation processing, then, the judgement unit 43 reads out operation which is next to the operation of turning off the coagulation mode of the electrocautery apparatus 13 on a time-series basis and judges whether or not the readout operation is operation of turning on the shadowless lights 3 (step S13). In a case where the readout operation is not the operation of turning on the shadowless lights 3 (N), the judgement unit 43 reads out operation which is the next on a time-series basis and executes step S13 again. In a case where the readout operation is operation of turning on the shadowless lights 3 (Y), settings of operation within a certain time period before and after the operation of turning on the shadowless lights 3 are collectively registered as collective settings for the scene of “start of the surgery” (step S14). More specifically, the judgement unit 43 judges operation of turning on the shadowless lights 3 (operation time point: 9:10:05), operation of starting recording to the recorder 17 (operation time point: 9:10:14), operation of switching a mode of the electrocautery apparatus 13 from the dissection mode to a bipolar dissection mode (9:10:20) and operation of setting a dissection output value of the electrocautery apparatus 13 at 75 W (operation time point: 9:10:53) as operation corresponding to the scene of “start of the surgery”. Then, the processing unit 44 generates settings of turning on the shadowless lights 3, settings of starting recording to the recorder 17, settings of switching the mode of the electrocautery apparatus 13 from the dissection mode to the bipolar dissection mode, and settings of setting the dissection output value of the electrocautery apparatus 13 at 75 W in association with the scene of “start of the surgery” and records the settings in the recording unit 42.

In the setting value generation processing, then, the judgement unit 43 reads out operation next to the operation of setting the dissection output value of the electrocautery apparatus 13 at 75 W on a time-series basis and judges whether or not the readout operation is operation of starting air feeding to the insufflation apparatus 14 (step S15). In a case where the readout operation is not the operation of starting air feeding (N), the judgement unit 43 reads out operation which is next on a time-series basis and executes step S15 again. In a case where the readout operation is the operation of starting air feeding (Y), settings of operation within a certain time period before and after the operation of starting air feeding are collectively registered as collective settings for the scene of “insertion of an endoscope” (step S16). More specifically, the judgement unit 43 judges operation of starting air feeding to the insufflation apparatus 14 (operation time point: 9:35:35), operation of turning on a light source of the video system center 15 (operation time point: 9:36:02), operation of switching a mode of the electrocautery apparatus 13 from the coagulation mode to a soft coagulation mode (operation time point: 9:37:17), and operation of setting a coagulation output value of the electrocautery apparatus 13 at 100 W (operation time point: 9:37:41) as operation corresponding to the scene of “insertion of an endoscope”. Then, the processing unit 44 generates settings of starting air feeding to the insufflation apparatus 14, settings of turning on the light source of the video system center 15, settings of switching the mode of the electrocautery apparatus 13 from the coagulation mode to the soft coagulation mode, and settings of setting the coagulation output value of the electrocautery apparatus 13 at 100 W in association with the scene of “insertion of an endoscope” and records the settings in the recording unit 42.

In the setting value generation processing, then, the judgement unit 43 reads out operation which is next to the operation of setting the coagulation output value of the electrocautery apparatus 13 at 100 W on a time-series basis and judges whether or not the readout operation is operation of stopping air feeding to the insufflation apparatus 14 (step S17). In a case where the readout operation is not the operation of stopping air feeding (N), the judgement unit 43 reads out operation which is next on a time-series basis and executes step S17 again. In a case where the readout operation is the operation of stopping air feeding (Y), settings of operation within a certain time period before and after the operation of stopping air feeding are collectively registered as collective settings for the scene of “closure” (step S18). More specifically, the judgement unit 43 judges operation of turning off the light source of the video system center 15 (operation time point: 12:52:10) and operation of stopping air feeding to the insufflation apparatus 14 (operation time point: 12:52:56) as operation corresponding to the scene of “closure”. Then, the processing unit 44 generates settings of turning off the light source of the video system center 15 and settings of stopping air feeding to the insufflation apparatus 14 in association with the scene of “closure” and records the settings in the recording unit 42.

In the setting value generation processing, then, the judgement unit 43 reads out operation which is next to the operation of turning off the light source of the video system center 15 on a time-series basis and judges whether or not the readout operation is operation of turning off the shadowless lights 3 (step S19). In a case where the readout operation is not the operation of turning off the shadowless lights 3 (N), the judgement unit 43 reads out operation which is next on a time-series basis and executes step S19 again. In a case where the readout operation is the operation of turning off the shadowless lights 3 (Y), settings of operation within a certain time period before and after the operation of turning off the shadowless lights 3 are collectively registered as collective settings for the scene of “end of the surgery” (step S20). More specifically, the judgement unit 43 judges that operation of stopping recording to the recorder 17 (operation time point: 13:12:46) and operation of turning off the shadowless lights 3 (operation time point: 13:13:11) as operation corresponding to the scene of “end of the surgery”. Then, the processing unit 44 generates settings of stopping recording to the recorder 17 and settings of turning off the shadowless lights 3 in association with the scene of “end of the surgery” and records the settings in the recording unit 42.

In the setting value generation processing, then, the judgement unit 43 reads out operation next to the operation of turning off the shadowless lights 3 on a time-series basis and judges whether or not the readout operation is operation of logging off from the centralized controller 22 (step S21). In a case where the readout operation is not the logoff operation (N), the judgement unit 43 reads out operation which is next on a time-series basis and executes step S21 again. In a case where the readout operation is the logoff operation (Y), the setting value generation processing is finished.

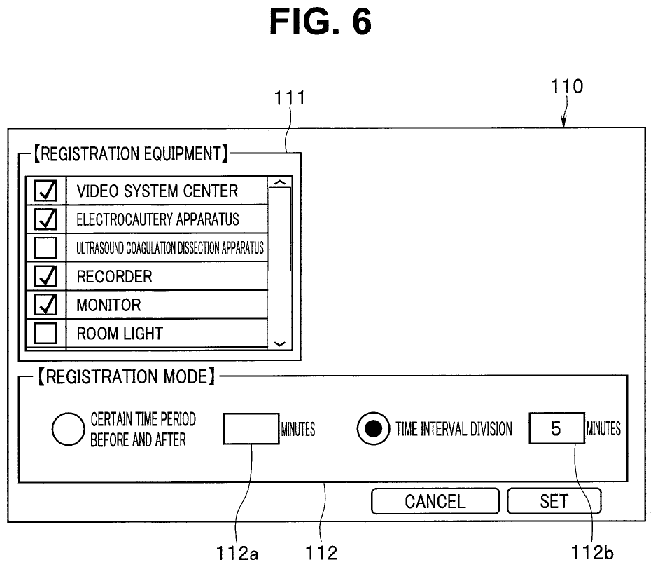

Note that in the above-described setting value generation processing, among a plurality of kinds of operation illustrated in FIG. 6, operation of setting the coagulation output value of the electrocautery apparatus 13 at 100 W (operation time point: 9:37:41) which is the previous operation of the operation of setting the dissection output value of the electrocautery apparatus 13 at 90 W (operation time point: 10:03:15) on a time-series basis is judged as the operation corresponding to the scene of “closure” by the judgement unit 43. Further, operation of turning off the light source of the video system center 15 (operation time point: 12:52:10) which is the subsequent processing of the operation of setting the dissection output value at 90 W on a time-series basis is judged as the operation corresponding to the scene of “end of the surgery” by the judgement unit 43. However, the operation of setting the dissection output value at 90 W itself is judged as neither the operation corresponding to the scene of “closure” nor the operation corresponding to the scene of “end of the surgery” by the judgement unit 43. The judgement is made because the operation of setting the dissection output value at 90 W does not satisfy both a condition that the operation is performed within a certain time period before and after the operation of starting air feeding (operation time point: 9:35:35) associated with the scene of “closure” and a condition that the operation is performed within a certain time period before and after the operation of stopping air feeding (operation time point: 12:52:56) associated with the scene of “end of the surgery”.

Description has been provided above using an example of a case where the judgement unit 43 performs scene correspondence judgement using the above-described first judgement method. In a case where the judgement unit 43 performs scene correspondence judgement using the above-described second judgement method, results which are the same as the results obtained by the above-described setting value generation processing can be obtained if the above-described predetermined time period is set at, for example, five minutes.

### (Setting Screen)

First to fourth setting screens relating to the setting value generation processing among setting screens to be displayed at the display unit of the operation panel apparatus 21 will be described next. The first setting screen will be described first with reference to FIG. 6. FIG. 6 illustrates a first setting screen 110 which is an example of the setting screen for setting a registration condition for registering collective settings. The first setting screen 110 includes an area 111 for selecting equipment for which registration is to be performed, and an area 112 for selecting a registration mode.

The area 111 is an area for selecting equipment for which collective settings are to be registered. In the present embodiment, a plurality of pieces of controlled equipment can be selected as controlled equipment for which the processing unit 44 generates setting values. In other words, in the setting value generation processing, only operation of the equipment selected in the area 111 is extracted from the operation log information and is set as a target which is to be judged by the judgement unit 43 and for which setting values are to be generated by the processing unit 44. The video system center 15, the electrocautery apparatus 13, the recorder 17 and the display apparatus 19 (described as a monitor in FIG. 6) are selected within a range of the area 111 illustrated in FIG. 6, and the ultrasound coagulation dissection apparatus 10 and the room light 7 are not selected. In the present case, in the setting value generation processing, operation of the ultrasound coagulation dissection apparatus 10 and the room light 7 is neither extracted from the operation log information nor set as a target which is to be judged by the judgement unit 43 and for which setting values are to be generated by the processing unit 44.

The area 112 is an area for selecting the registration mode. In the example illustrated in FIG. 6, a mode of a “certain time period before and after” and a mode of “time interval division” can be selected. The mode of the “certain time period before and after” is a mode corresponding to the above-described first judgement method. A field 112a within the area 112 is a field in which a set time period for the mode of the “certain time period before and after” is to be inputted. In a case where the mode of the “certain time period before and after” is selected, the judgement unit 43 judges that a plurality of kinds of operation which are performed within a time period from a time point the above-described set time period before the trigger time point until a time point the above-described set time period after the trigger time point as the operation corresponding to the scene.

The mode of the “time interval division” is a mode corresponding to the above-described second judgement method. A field 112b within the area 112 is a field in which a set time period of the mode of the “time interval division” is to be inputted. In a case where the mode of the “time interval division” is selected, the judgement unit 43 judges that a plurality of kinds of operation which are continuous with the trigger operation on a time-series basis and for which an interval between two kinds of operation which are adjacent to each other on a time-series basis is equal to or less than the above-described set time period, as the operation corresponding to the scene.

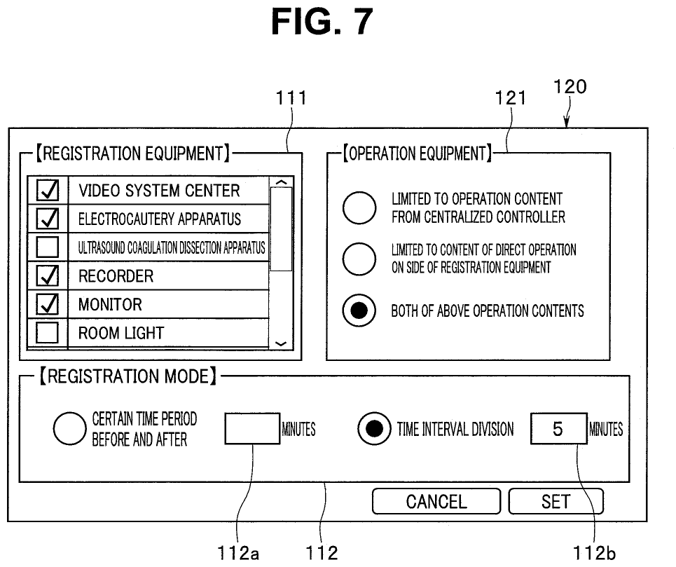

The second setting screen will be described next with reference to FIG. 7. FIG. 7 illustrates a second setting screen 120 which is another example of the setting screen for setting a registration condition for registering collective settings. The second setting screen 120 includes the areas 111 and 112 in a similar manner to the first setting screen 110 illustrated in FIG. 6. The second setting screen 120 further includes an area 121 for selecting equipment to be operated.

The area 121 is an area for selecting equipment to be operated for which collective settings are to be registered. In the present embodiment, a plurality of kinds of operation include indirect operation performed on a plurality of pieces of controlled equipment from the operation panel apparatus 21 and direct operation performed at the plurality of pieces of controlled equipment, and at least one of the indirect operation or the direct operation is selectable as the plurality of kinds of operation for which setting values are to be generated by the processing unit 44. In the example illustrated in FIG. 7, it is possible to select whether operation is “limited to operation content from the centralized controller 22” or “limited to content of direct operation on a side of equipment for which registration is to be performed”, or “both of the above-described operation content” is set. In a case where “limited to operation content from the centralized controller 22” is selected, a plurality of kinds of operation for which setting values are to be generated by the processing unit 44 are limited to indirect operation. In a case where “limited to operation content of direct operation on a side of equipment for which registration is to be performed” is selected, a plurality of kinds of operation for which setting values are to be generated by the processing unit 44 are limited to direct operation. In a case where “both of the above-described operation content” is selected, both indirect operation and direct operation are set as targets for a plurality of kinds of operation for which setting values are to be generated by the processing unit 44.

Note that the first setting screen 110 and the second setting screen 120 may be able to be selected, for example, on other setting screens which are not illustrated.

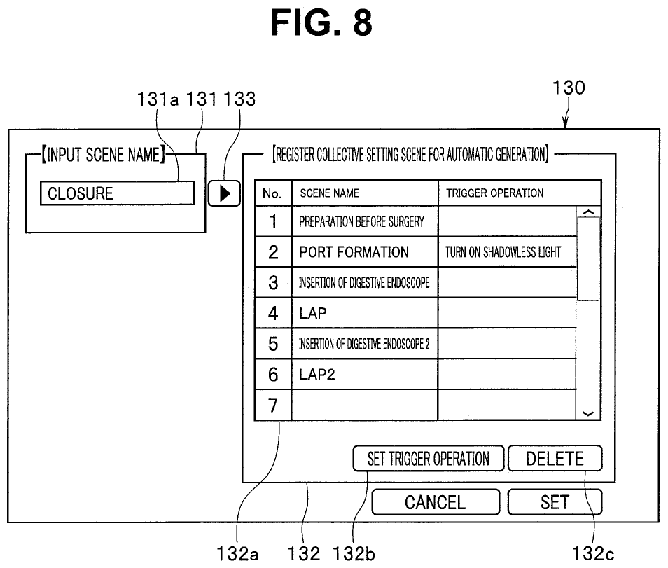

The third setting screen will be described next with reference to FIG. 8. FIG. 8 illustrates the third setting screen 130 which is an example of the setting screen for setting a scene condition for registering collective settings. The third setting screen 130 includes an area 131 for inputting a name of the scene and an area 132 indicating a list of scenes and a list of trigger operation. A field 131a within the area 131 is an input field of a name of the scene, which allows the user to freely input the name of the scene. Further, after the name of the scene is inputted, the scene inputted to the field 131a can be registered by a button 133 located between the area 131 and the area 132 being touched, so that the inputted scene is reflected in a table 132a within the area 132. Note that the registered scene can be deleted by a “delete” button 132c within the area 132 being touched in a state where the name of the scene is touched and selected.

Further, fields of trigger operation corresponding to respective scenes are provided in the table 132a, and the trigger operation corresponding to the respective scenes is displayed in the fields. Further, the trigger operation corresponding to the respective scenes can be registered by a “set trigger operation” button 132b located within the area 132 being touched in a state where the field is touched and selected. The trigger operation can be registered, for example, using the fourth setting screen which will be described later.

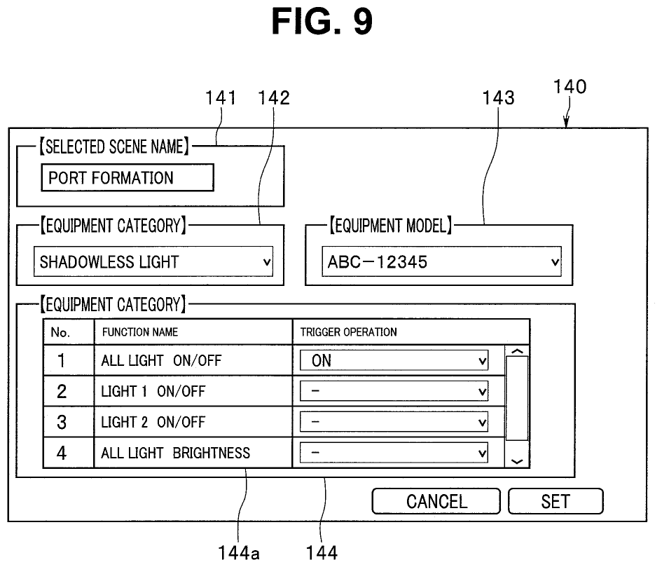

The fourth setting screen will be described next with reference to FIG. 9. FIG. 9 illustrates the fourth setting screen 140 which is an example of the setting screen for setting trigger operation. The fourth setting screen 140 includes an area 141 for displaying a name of the selected scene, an area 142 for selecting an equipment category, an area 143 for selecting an equipment model, and an area 144 for selecting trigger operation.

Here, description will be provided using an example of a setting screen which appears by the “set trigger operation” button 132b being touched in a state where a field of trigger operation of port formation is touched and selected on the third setting screen 130 illustrated in FIG. 8. The name of the scene selected on the third setting screen 130 is displayed in the area 141.

The area 142 is an area for selecting an equipment category for which trigger operation is to be performed. The area 143 is an area for selecting an equipment model for which trigger operation is to be performed. A corresponding equipment model can be touched and selected if the equipment category is touched and selected.

The area 144 is an area for selecting trigger operation of the equipment selected in the area 143. If the equipment model is selected, functions of the equipment are displayed in a table 144a within the area 144. Further, fields of trigger operation corresponding to respective functions are provided in the table 144a, which allows the user to touch the field to select trigger operation. Further, the trigger operation can be registered by a set button located in a lower part of the fourth setting screen 140 being touched in a state where the trigger operation is selected.

### (Operation and Effects)

Operation and effects of the centralized controller 22 according to the present embodiment will be described next. In the present embodiment, the judgement unit 43 performs scene correspondence judgement for each scene of the surgery, and the processing unit 44 generates at least part of setting values of a plurality of pieces of controlled equipment in association with the scenes of the surgery from a plurality of kinds of operation which are judged as operation corresponding to the scenes through the scene correspondence judgement and records the setting values in the recording unit 42. As a result, according to the present embodiment, it is possible to automatically generate setting values associated with the scenes of the surgery. As a result, according to the present embodiment, it is possible to eliminate the need or substantially reduce the work for associating scenes of the surgery with setting values, so that it is possible to reduce a time period required for registration work and editing work of setting values.

Further, in the present embodiment, for example, equipment for which collective settings are to be registered, that is, controlled equipment for which setting values are to be generated by the processing unit 44 can be selected through, for example, the first setting screen 110 illustrated in FIG. 6 or the second setting screen 120 illustrated in FIG. 7. In other words, in the present embodiment, it is possible to select controlled equipment for which setting values are not generated by the processing unit 44. As a result, according to the present embodiment, it is possible to eliminate the need of work of deleting unnecessary setting values, so that it is possible to generate setting values which more closely conform to intentions of the user.

Further, in the present embodiment, for example, operation equipment for which collective settings are to be registered can be set, for example, through the third setting screen 130 illustrated in FIG. 8, so that it is possible to select indirect operation or direct operation as the plurality of kinds of operation for which the processing unit 44 generates setting values. Particularly, in a case where the operation for which collective settings are to be registered is limited to operation content from the centralized controller 22, that is, in a case where a plurality of kinds of operation for which the processing unit 44 generates setting values are limited to indirect operation, it is possible to impose a limitation so as to prevent setting values from being generated through irregular operation during the surgery. The irregular operation includes, for example, change of output values, or the like, by the electrocautery apparatus 13 being directly operated during the surgery. As a result, according to the present embodiment, it is possible to eliminate the need of work of deleting unnecessary setting values, so that it is possible to generate setting values which more closely conform to intentions of the user.

Note that in the present embodiment, the setting value generation processing at the judgement unit 43 and the processing unit 44 is performed on a series of operation from operation of logging in to the centralized controller 22 to operation of logging out from the centralized controller 22. In other words, in the present embodiment, setting values of the controlled equipment are generated through a plurality of kinds of operation in certain specific procedure by a certain specific surgeon. As a result, according to the present embodiment, it is possible to eliminate the need or substantially reduce work of associating surgeons and procedure with setting values, so that it is possible to reduce a time period required for registration work and editing work of the setting values.

### Second Embodiment

### (Configuration of Centralized Controller)

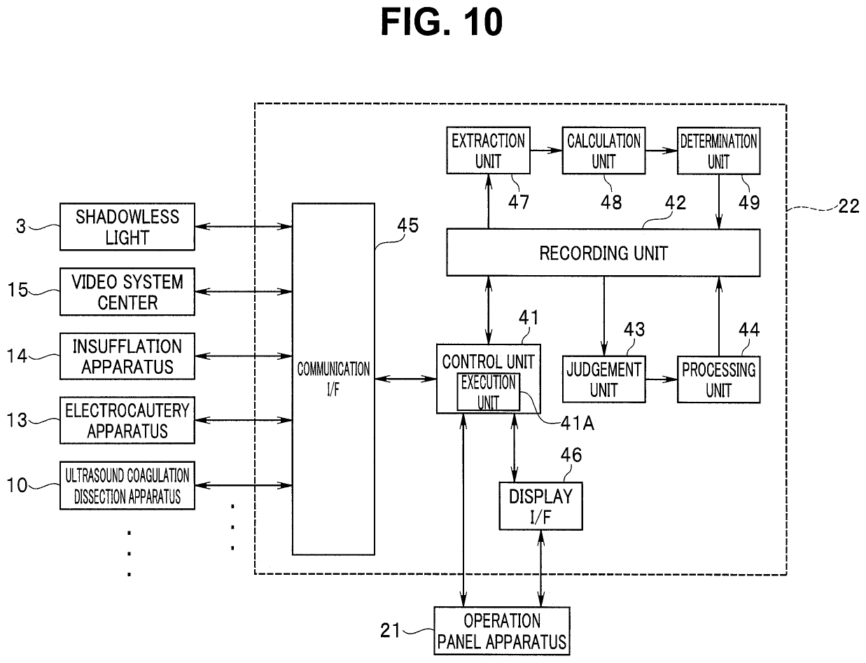

A centralized controller according to a second embodiment of the present invention will be described next. A configuration of the centralized controller will be described first. FIG. 10 is a functional block diagram illustrating the configuration of the centralized controller.

In the present embodiment, the centralized controller 22 includes the control unit 41, the recording unit 42, the judgement unit 43, the processing unit 44, the communication I/F 45 and the display I/F 46 in a similar manner to the first embodiment. Functions and operation of the control unit 41, the recording unit 42, the judgement unit 43, the processing unit 44, the communication I/F 45 and the display I/F 46 are similar to the functions and the operation in the first embodiment except operation of the judgement unit 43 which will be described later.

In the present embodiment, the centralized controller 22 further includes an extraction unit 47, a calculation unit 48 and a determination unit 49. As described in the first embodiment, the recording unit 42 records information on a plurality of kinds of operation as one piece of operation log information for each piece of specific information included in the login information. The extraction unit 47 reads out a plurality of pieces of operation log information from the recording unit 42 for each piece of specific information. Specifically, the specific information includes a name of the surgeon and a name of the procedure. The extraction unit 47 may read out the operation log information for each kind of procedure or may read out the operation log information for each kind of procedure by the same surgeon.

The operation log information includes a plurality of aggregates including a plurality of kinds of operation which are continuous on a time-series basis and for which an interval between two kinds of operation which are adjacent to each other on a time-series basis is equal to or less than a predetermined time period. The extraction unit 47 extracts a plurality of aggregates which are included in different pieces of operation log information and to which the same operation belongs as one scene candidate aggregate from the plurality of pieces of the readout operation log information and extracts a plurality of scene candidate aggregates.

Here, a case will be assumed where the extraction unit 47 reads out first to ninth operation log information as a plurality of pieces of operation log information, and the first to the ninth operation log information respectively include aggregates to which operation of logging in to the centralized controller 22 belongs. In the present case, the extraction unit 47 extracts the above-described respective aggregates of the first to the ninth operation log information as one scene candidate aggregate. Further, a case will be assumed where the extraction unit 47 reads out the first to the ninth operation log information as a plurality of pieces of operation log information, and the first to the ninth operation log information respectively include other aggregates to which operation of turning on the shadowless lights 3 belongs. In the present case, the extraction unit 47 extracts the above-described respective other aggregates of the first to the ninth operation log information as another one scene candidate aggregate.

Hereinafter, each of a plurality of kinds of operation included in the scene candidate aggregate will be referred to as a trigger operation candidate. The calculation unit 48 counts the number of trigger operation candidates corresponding to the same operation belonging to the scene candidate aggregate for each scene candidate aggregate. Here, a case will be assumed where the extraction unit 47 reads out the first to the ninth operation log information as the plurality of pieces of operation log information and extracts an aggregate to which operation of stopping air feeding to the insufflation apparatus 14 belongs, included in each of the first to the ninth operation log information, as one scene candidate aggregate. In the present case, a case will be further assumed where while the operation of turning off the light source of the video system center 15 belongs to an aggregate to which the operation of stopping air feeding included in each of the first to the eighth operation log information belongs, the operation of turning off the light source of the video system center 15 does not belong to an aggregate to which the operation of stopping air feeding included in the ninth operation log information belongs. In the present case, the calculation unit 48 counts the number of times of the operation of stopping air feeding and the operation of turning off the light source which are trigger operation candidates corresponding to the same operation belonging to the scene candidate aggregate extracted by the extraction unit 47. In the example, the number of times of the operation of stopping air feeding is nine, and the number of times of the operation of turning off the light source is eight.

The determination unit 49 determines one kind of trigger operation associated with the scene candidate aggregate from a plurality of trigger operation candidates belonging to the scene candidate aggregate based on the number counted by the calculation unit 48 for each scene candidate aggregate and records the trigger operation in the recording unit 42 in association with specific information. In the present embodiment, the calculation unit 48 calculates a ratio of the number of the trigger operation candidates counted by the calculation unit 48 with respect to the number of pieces of the readout operation log information as an operation rate of the trigger operation candidates. The determination unit 49 determines a trigger operation candidate with the highest operation rate as trigger operation. Description will be provided using an example of a case where an aggregate to which the operation of stopping air feeding to the insufflation apparatus 14 belongs, included in each of the first to the ninth operation log information is extracted as one scene candidate aggregate described above. An operation rate of the operation of stopping air feeding is 100%, and an operation rate of the operation of turning off the light source is 89%. In the example, the determination unit 49 determines the operation of stopping air feeding as trigger operation.

Further, the scene candidate aggregate has correspondence relationship with scenes of the surgery. The determination unit 49 records the trigger operation in the recording unit 42 in association with a scene of the surgery corresponding to the scene candidate aggregate with which the trigger operation is associated. The scene candidate aggregate may have correspondence relationship with names of scenes of the surgery or may have correspondence relationship with order of scenes of the surgery. In the former case, the determination unit 49 records the trigger operation in association with the names of scenes of the surgery such as preparation before the surgery. In the latter case, the determination unit 49 records the trigger operation in association with the order of scenes of the surgery.

Hereinafter, a series of processing at the extraction unit 47, the calculation unit 48 and the determination unit 49 for determining the trigger operation will be referred to as trigger operation determination processing.

In the present embodiment, the judgement unit 43 reads out trigger operation associated with the scene from the recording unit 42 for each scene of the surgery and performs scene correspondence judgement based on a trigger time point which is a time point at which the readout trigger operation is performed. In the present embodiment, particularly, the judgement unit 43 reads out a plurality of pieces of operation log information from the recording unit 42 for each piece of specific information, extracts a plurality of kinds of operation having correspondence relationship and other than the trigger operation from different pieces of operation log information as one non-trigger operation candidate and extracts a plurality of non-trigger operation candidates. The scene correspondence judgement is performed for each kind of trigger operation and for each non-trigger operation candidate.

The above-described correspondence relationship may be relationship in which content of operation is completely the same or may be relationship in which content of operation is similar. The relationship in which content of operation is completely the same is, for example, relationship such that all the coagulation output values become the same in operation of setting the coagulation output value at the electrocautery apparatus 13. The relationship in which content of operation is similar is, for example, relationship such that values of the coagulation output values are statistically approximated in operation of setting the coagulation output value at the electrocautery apparatus 13. Examples of a method for extracting a plurality of kinds of operation in which content of operation is similar as one non-trigger operation candidate can include, for example, a statistical method or a method using artificial intelligence.

Further, in a case where a plurality of kinds of operation in which content of operation is completely the same is extracted as one non-trigger operation candidate, the judgement unit 43 may extract a plurality of the same kinds of operation other than the trigger operation from different pieces of operation log information as one operation candidate, extract a plurality of operation candidates, calculate a ratio of the number of the plurality of the same kinds of operation corresponding to the operation candidate with respect to the number of pieces of readout operation log information for each operation candidate, and extract operation for which the ratio is equal to or higher than a predetermined threshold as a non-trigger operation candidate.

As described in the first embodiment, a screen for setting values of a plurality of pieces of controlled equipment for each scene of the surgery is displayed at the display unit of the operation panel apparatus 21. In the present embodiment, particularly, the user can give an instruction to execute the trigger operation determination processing of automatically determining trigger operation by, for example, touching a button for executing trigger operation determination. In a case where a signal which gives an instruction to execute the trigger operation determination processing is inputted from the operation panel apparatus 21, the control unit 41 controls the extraction unit 47, the calculation unit 48 and the determination unit 49 so that the trigger operation is determined.

Functions of the extraction unit 47, the calculation unit 48 and the determination unit 49 may be, for example, implemented with the processor 22A and the storage apparatus 22B illustrated in FIG. 3 in the first embodiment in a similar manner to other components of the centralized controller 22. Alternatively, the extraction unit 47, the calculation unit 48 and the determination unit 49 may be respectively configured with electronic circuits separate from other components of the centralized controller 22.

Further, functions of the storage unit which is not illustrated and which will be described later are, for example, implemented with a rewritable storage device such as a RAM among the storage apparatus 22B.

### (Trigger Operation Determination Processing)

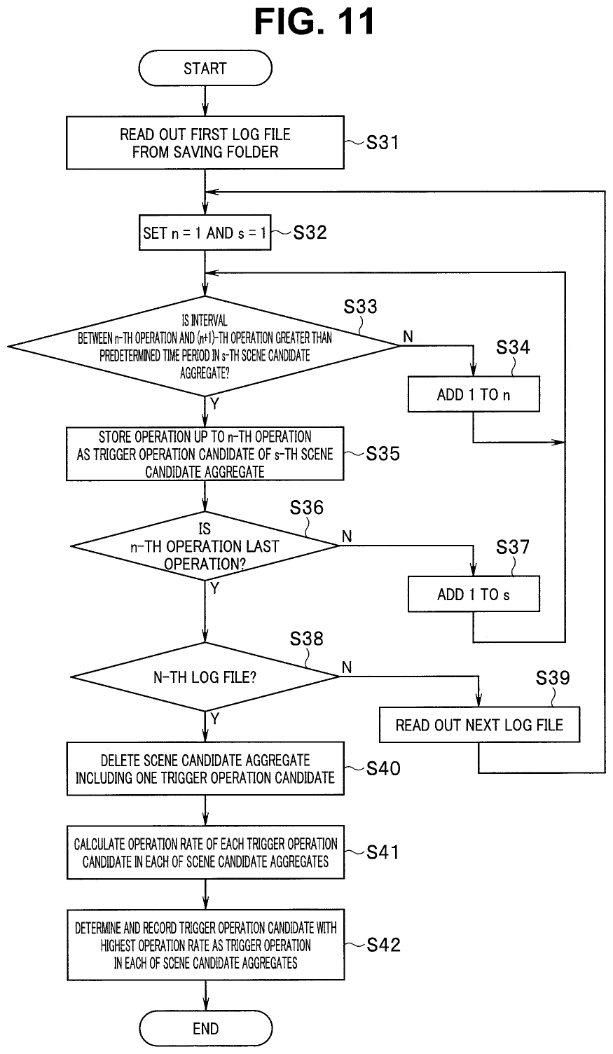

The trigger operation determination processing, that is, operation of the extraction unit 47, the calculation unit 48 and the determination unit 49 will be described next with reference to FIG. 11. FIG. 11 is a flowchart illustrating the trigger operation determination processing. Here, description will be provided using an example of a case where the trigger operation determination processing is performed for each kind of procedure by the same surgeon.

As described in the first embodiment, the operation log information is stored in a non-volatile rewritable storage apparatus which configures the recording unit 42, as a log file. It is assumed here that the log file is a plurality of files divided for each name of the surgeons and each name of the procedure, and each of a plurality of files is stored in a corresponding saving folder. Here, N log files with the same surgeon and the same procedure will be referred to as first to N-th log files for convenience sake. In the trigger operation determination processing, the first to the N-th log files are sequentially read out.

As illustrated in FIG. 11, in the trigger operation determination processing, first, the extraction unit 47 reads out the first log file from the saving folder in which N log files with the same surgeon and the same procedure are stored (step S31). Then, the extraction unit 47 defines variables n and s for which initial values are respectively integers of 1 (step S32).

Then, the extraction unit 47 determines whether or not an interval between n-th operation and (n+1)-th operation on a time-series basis in an s-th scene candidate aggregate on a time-series basis among a plurality of kinds of operation included in the readout log file is greater than a predetermined time period (step S33). In a case where the interval is not greater than the predetermined time period (N), the extraction unit 47 adds 1 to the variable n (step S34), and the processing returns to step S33. In a case where the interval is greater than the predetermined time period (Y), the extraction unit 47 stores operation up to the n-th operation in a storage unit which is not illustrated as trigger operation candidates of the s-th scene candidate aggregate (step S35).

Then, the extraction unit 47 determines whether or not the n-th operation is the last operation on a time-series basis in the readout log file (step S36). In a case where the n-th operation is not the last operation (N), the extraction unit 47 adds 1 to the variable s (step S37), and the processing returns to step S33. In a case where the n-th operation is the last operation (Y), the extraction unit 47 determines whether or not the readout log file is an N-th log file (step S38). In a case where the readout log file is not the N-th log file (N), the extraction unit 47 reads out the next log file (step S39), and the processing returns to step S32. In a case where the readout log file is the N-th log file (Y), the extraction unit 47 deletes a scene candidate aggregate including one trigger operation candidate among a plurality of scene candidate aggregates from a storage unit which is not illustrated (step S40).

In the trigger operation determination processing, the calculation unit 48 then calculates an operation rate of each trigger operation candidate for each of a plurality of scene candidate aggregates (step S41).

In the trigger operation determination processing, the determination unit 49 then determines a trigger operation candidate with the highest operation rate in each of the plurality of scene candidate aggregates as trigger operation corresponding to the scene candidate aggregate, records the determined trigger operation in the recording unit 42 (step S42), and the trigger operation determination processing is finished.

Note that description has been provided above using an example of a case where the trigger operation determination processing is performed for each kind of procedure by the same surgeon. The trigger operation determination processing may be performed for each kind of the procedure. In other words, the trigger operation determination processing may be performed without a surgeon being taken into account. In the present case, the extraction unit 47 reads out log files from one or more saving folders in which N log files with the same procedure are stored instead of reading out log files from a saving folder in which N log files with the same surgeon and the same procedure are stored.

Further, in step S40, a scene candidate aggregate including one trigger operation candidate is excluded as, so-called an “outlier” or an “abnormal value”. It is considered that the “outlier” or the “abnormal value” corresponds to the irregular operation described in the first embodiment. However, in step S40, a trigger operation candidate recognized as the “outlier” or the “abnormal value” may be excluded using a statistical method or artificial intelligence.

### (Setting Value Generation Processing)

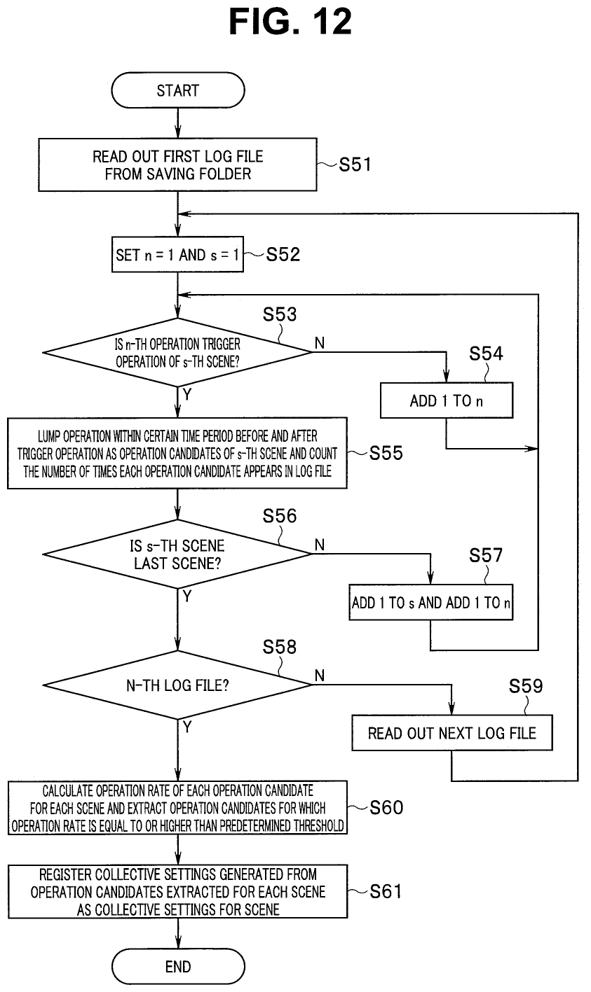

Setting value generation processing in the present embodiment will be described next with reference to FIG. 12. FIG. 12 is a flowchart illustrating the setting value generation processing. Here, description will be provided using an example of a case where the setting value generation processing is performed for each kind of procedure by the same surgeon. In the setting value generation processing, the first to the N-th log files are sequentially read out in a similar manner to the trigger operation determination processing.

As illustrated in FIG. 12, in the setting value generation processing, the judgement unit 43 first reads out the first log file from the saving folder in which N log files with the same surgeon and the same procedure are stored (step S51). Then, the judgement unit 43 defines variables n and s for which initial values are respectively integers of 1 (step S52).

Then, the judgement unit 43 determines whether or not n-th operation on a time-series basis is trigger operation of an s-th scene on a time-series basis (step S53). In a case where the n-th operation is not trigger operation (N), the judgement unit 43 adds 1 to the variable n (step S54), and the processing returns to step S53. In a case where the n-th operation is trigger operation (Y), the judgement unit 43 lumps operation within a certain time period before and after the trigger operation as operation candidates for the s-th scene and counts the number of times each operation candidate appears in the log file (step S55).

Then, the judgement unit 43 determines whether or not the s-th scene is the last scene on a time-series basis in the readout log file (step S56). In a case where the s-th scene is not the last scene (N), the judgement unit 43 adds 1 to the variable s, adds 1 to the variable n (step S57), and the processing returns to step S53. In a case where the s-th scene is the last scene (Y), the judgement unit 43 determines whether or not the readout log file is the N-th log file (step S58). In a case where the readout log file is not the N-th log file (N), the judgement unit 43 reads out the next log file (step S59), and the processing returns to step S52. In a case where the readout log file is the N-th log file (Y), the judgement unit 43 calculates an operation rate of each operation candidate for each scene and extracts an operation candidate for which the operation rate is equal to or higher than a predetermined threshold (step S60). The judgement unit 43 calculates a ratio of the number of the same operation candidates with respect to the number of readout log files as an operation rate of the operation candidates. The threshold is, for example, 80%.

In the setting value generation processing, then, settings generated from the operation candidates extracted for each scene in step S60 are registered as collective settings of the scene (step S61). More specifically, the judgement unit 43 judges that the operation candidates extracted for each scene in step S60 as operation corresponding to the scene, and the processing unit 44 generates setting values of a plurality of pieces of controlled equipment from the extracted operation candidates and records the setting values in the recording unit 42. In a case where collective settings are registered for all scenes, the setting value generation processing is finished.

Note that description has been provided above using an example of a case where the setting value generation processing is performed for each kind of procedure by the same surgeon. The setting value generation processing may be performed for each kind of procedure. In other words, the setting value generation processing may be performed without a surgeon being taking into account. In the present case, the judgement unit 43 reads out log files from one or more saving folders in which N log files with the same procedure are stored instead of reading out log files from saving folders in which N log files with the same surgeon and the same procedure are stored.

Further, in step S55, the judgement unit 43 lumps operation as a plurality of operation candidates using a method similar to the first judgement method described in the first embodiment. However, in step S55, the judgement unit 43 may lump operation as a plurality of operation candidates using a method similar to the second judgement method described in the first embodiment.

Further, in step S60, the judgement unit 43 extracts operation candidates for which the operation rate is equal to or higher than a predetermined threshold. However, in step S60, the judgement unit 43 may extract a plurality of kinds of operation having relationship in which content of operation is similar as operation candidates using a statistical method such as standard deviation or may extract a plurality of kinds of operation having relationship in which content of operation is similar as operation candidates using artificial intelligence.

### (Operation and Effects)

Operation and effects specific to the present embodiment will be described next. In the present embodiment, operation log information is read out for each piece of specific information of the login information, and trigger operation is determined from the readout operation log information. As a result, according to the present embodiment, it is possible to eliminate the need of work of setting trigger operation, so that it is possible to reduce a time period required for registration work and editing work of the setting values.

Further, in the present embodiment, the setting value generation processing is performed based on the trigger operation determined through the trigger operation determination processing, the operation log information is read out for each piece of specific information of the login information, and the setting value generation processing is executed using the readout operation log information. As a result, according to the present embodiment, it is possible to generate standard setting values for each surgeon or for each kind of procedure.

Further, in the present embodiment, the judgement unit 43 calculates an operation rate of each operation candidate for each scene and extracts operation candidates for which the operation rate is equal to or higher than a predetermined threshold (step S60 in FIG. 12). It is considered that operation candidates for which the operation rate is less than the predetermined threshold correspond to the irregular operation described in the first embodiment or erroneous operation deviating from intentions of the user. As a result, according to the present embodiment, it is possible to prevent setting values from being generated through irregular operation or erroneous operation. As a result, according to the present embodiment, it is possible to eliminate the need of work of deleting unnecessary setting values and generate setting values which more closely conform to the intentions of the user.

Other configurations, operation and effects in the present embodiment are similar to the configurations, operation and effects in the first embodiment.

The present invention is not limited to the above-described embodiments, and various changes, modifications, or the like, can be made within a scope not deviating from the gist of the present invention. For example, in the second embodiment, the setting value generation processing may be performed based on trigger operation set by the user in place of the trigger operation determined through the trigger operation determination processing.

What is claimed is:

1. A centralized control apparatus comprising a processor,
the processor being configured to
record information on a plurality of kinds of operation performed on a plurality of pieces of controlled equipment along with time points at which the plurality of kinds of operation are performed and record the information on the plurality of kinds of operation as one piece of operation log information for each piece of specific information which specifies at least one of a name of a surgeon or a name of procedure,
read out the piece of the operation log information in plurality from a recording result of the operation log information for each piece of the specific information, extract an aggregate of a plurality of kinds of operation which are included in different pieces of the operation log information and to which same operation belongs as one scene candidate aggregate and extract the scene candidate aggregate in plurality,
in a case where each of the plurality of kinds of operation belonging to the scene candidate aggregate is set as each of trigger operation candidates from an extraction result of the scene candidate aggregate, count, for each of the scene candidate aggregate in plurality, a number of the trigger operation candidates which correspond to the same operation belonging to the scene candidate aggregate,
determine, for each of the scene candidate aggregate in plurality, one piece of trigger operation associated with the scene candidate aggregate from a plurality of the trigger operation candidates belonging to the scene candidate aggregate based on the counted number of the trigger operation candidates and record the trigger operation as the operation log information in association with the specific information, and
read out setting values of one or more pieces of controlled equipment which are setting targets based on the operation log information for each of scenes of a surgery defined in advance and collectively set the setting values at the one or more pieces of controlled equipment.

2. The centralized control apparatus according to claim 1, wherein
the processor calculates a ratio of the number of the trigger operation candidates with respect to a number of pieces of the readout operation log information as an operation rate of the trigger operation candidates, and
determines a trigger operation candidate with the highest operation rate as the trigger operation.

3. The centralized control apparatus according to claim 2, wherein
the processor is further configured to read out the information on the plurality of kinds of operation and perform, for each of the scenes of the surgery, scene correspondence judgement as to whether or not each of the plurality of kinds of operation is operation corresponding to the scene which is operation of the scene,
the scene candidate aggregate has correspondence relationship with the scenes of the surgery,
the processor records the trigger operation in association with the scenes of the surgery corresponding to the scene candidate aggregate with which the trigger operation is associated, and
reads out, for each of the scenes of the surgery, the trigger operation associated with the scene and performs the scene correspondence judgement based on a trigger time point which is a time point at which the readout trigger operation is performed.

4. The centralized control apparatus according to claim 3, wherein
the processor reads out the piece of the operation log information in plurality for each piece of the specific information, extracts the plurality of kinds of operation having correspondence relationship and other than the trigger operation as one non-trigger operation candidate from different pieces of the operation log information and extracts the non-trigger operation candidate in plurality, and
the scene correspondence judgement is performed for each kind of the trigger operation and for each of the non-trigger operation candidate.

5. The centralized control apparatus according to claim 4, wherein
the processor extracts the plurality of kinds of operation which are same and other than the trigger operation as one operation candidate from different pieces of the operation log information, extracts the operation candidate in plurality, calculates, for each of the operation candidate in plurality, a ratio of a number of the plurality of kinds of operation which are the same and which correspond to the operation candidate with respect to the number of pieces of the readout operation log information and extracts the operation candidate for which the ratio is equal to or higher than a predetermined threshold as the non-trigger operation candidate.

6. The centralized control apparatus according to claim 1, wherein
the processor is further configured to read out the information on the plurality of kinds of operation and perform, for each of the scenes of the surgery, scene correspondence judgment as to whether or not each of the plurality of kinds of operation is operation corresponding to the scene which is operation of the scene,
the plurality of kinds of operation include a plurality of kinds of trigger operation associated with the scenes of the surgery different from each other, and
the processor records the information on the plurality of kinds of operation along with time points at which the plurality of kinds of operation are performed, and
performs, for each of the scenes of the surgery, the scene correspondence judgement based on a trigger time point which is a time point at which operation associated with the scene among the plurality of kinds of trigger operation is performed.

7. The centralized control apparatus according to claim 6, wherein
the processor judges that the plurality of kinds of operation performed within a predetermined time period including the trigger time point as the operation corresponding to the scene.

8. The centralized control apparatus according to claim 6, wherein
the processor judges the plurality of kinds of operation which are continuous with the trigger operation on a time-series basis and for which an interval between two kinds of operation which are adjacent to each other on the time-series basis is equal to or less than a predetermined time period as the operation corresponding to the scene.

9. The centralized control apparatus according to claim 1, wherein
the processor is further configured to read out the information on the plurality of kinds of operation and perform, for each of the scenes of the surgery, scene correspondence judgement as to whether or not each of the plurality of kinds of operation is operation corresponding to the scene which is operation of the scene, and
generate and record at least part of setting values of the plurality of pieces of controlled equipment in association with the scenes of the surgery from the plurality of kinds of operation which are respectively judged as the operation corresponding to the scene through the scene correspondence judgement, and
the plurality of pieces of controlled equipment for which the processor generates the setting values are selectable.

10. The centralized control apparatus according to claim 1, wherein
the plurality of kinds of operation include indirect operation performed on the plurality of pieces of controlled equipment from an operation apparatus connected to the centralized control apparatus and direct operation performed at the plurality of pieces of controlled equipment, and
at least one of the indirect operation or the direct operation is selectable as the plurality of kinds of operation for which the processor generates the setting values.

11. The centralized control apparatus according to claim 1, wherein
the processor records the information on the plurality of kinds of operation in association with login information to the centralized control apparatus.

12. The centralized control apparatus according to claim 11, wherein
the login information includes the specific information which specifies at least one of the name of the surgeon or the name of the procedure, and
processing at the processor is performed for each piece of the specific information.

13. A setting method in which a centralized control apparatus sets setting values at a plurality of pieces of controlled equipment in accordance with scenes of a surgery, the setting method comprising:
recording information on a plurality of kinds of operation along with time points at which the plurality of kinds of operation are performed and recording the information on the plurality of kinds of operation as one piece of operation log information including a plurality of aggregates each including the plurality of kinds of operation which are continuous on a time-series basis and for which an interval between two kinds of operation which are adjacent to each other on the time-series basis is equal to or less than a predetermined time period, for each piece of specific information which specifies at least one of a name of a surgeon or a name of procedure;
reading out the piece of the operation log information in plurality for each piece of the specific information, extracting an aggregate of a plurality of kinds of operation which are included in different pieces of the operation log information and to which same operation belongs as one scene candidate aggregate and extracting the scene candidate aggregate in plurality;
in a case where each of the plurality of kinds of operation belonging to the scene candidate aggregate is set as each of trigger operation candidates, counting, for each of the scene candidate aggregate in plurality, a number of the trigger operation candidates which correspond to the same operation belonging to the scene candidate aggregate;
determining, for each of the scene candidate aggregate in plurality, one piece of trigger operation associated with the scene candidate aggregate from a plurality of the trigger operation candidates belonging to the scene candidate aggregate based on the counted number of the trigger operation candidates and recording the trigger operation as the operation log information in association with the specific information; and
reading out setting values of one or more pieces of controlled equipment which are setting targets based on the operation log information for each of scenes of the surgery defined in advance and collectively setting the setting values at the one or more pieces of controlled equipment.

14. A centralized control system comprising:
a plurality of pieces of controlled equipment; and
a processor to which the plurality of pieces of controlled equipment are connected,
the processor being configured to
record information on a plurality of kinds of operation along with time points at which the plurality of kinds of operation are performed and record the information on the plurality of kinds of operation as one piece of operation log information for each piece of specific information which specifies at least one of a name of a surgeon or a name of procedure,
read out the piece of the operation log information in plurality from a recording result of the operation log information for each piece of the specific information, extract an aggregate of a plurality of kinds of operation which are included in different pieces of the operation log information and to which same operation belongs as one scene candidate aggregate and extract the scene candidate aggregate in plurality,
in a case where each of the plurality of kinds of operation belonging to the scene candidate aggregate is set as each of trigger operation candidates from an extraction result of the scene candidate aggregate, count, for each of the scene candidate aggregate in plurality, a number of the trigger operation candidates which correspond to the same operation belonging to the scene candidate aggregate,
determine, for each of the scene candidate aggregate in plurality, one piece of trigger operation associated with the scene candidate aggregate from a plurality of the trigger operation candidates belonging to the scene candidate aggregate based on the counted number of the trigger operation candidates and record the trigger operation as the operation log information in association with the specific information, and
read out setting values of one or more pieces of controlled equipment which are setting targets based on the operation log information for each of scenes of a surgery defined in advance and collectively set the setting values at the one or more pieces of controlled equipment.
